(19) United States
(12) Patent Application Publication
(10) Pub. No.: US 2020/0108031 A1
(43) Pub. Date: Apr. 9, 2020

(54) Treating Tumors Using TTFields Combined with ABT-751

(71) Applicant: Novocure GmbH, Root D4 (CH)

(72) Inventors: Gerben BORST, Amsterdam (NL); Olaf van TELLINGEN, Zaandam (NL); Moshe GILADI, Moshav Herut (IL); Mark C. de GOOIJER, Landsmeer (NL); Paul L.G. SLANGEN, Amsterdam (NL)

(21) Appl. No.: 16/593,081

(22) Filed: Oct. 4, 2019

Related U.S. Application Data
(60) Provisional application No. 62/741,791, filed on Oct. 5, 2018.

(51) Int. Cl. A61K 31/18; A61P 35/00; A61N 1/36

(52) U.S. Cl. CPC A61K 31/18; A61N 1/36002; A61P 35/00

(57) ABSTRACT

Viability of cancer cells (e.g., glioblastoma cells) can be reduced by administering ABT-751 to the cancer cells and applying an alternating electric field with a frequency between 100 and 400 kHz (e.g., 200 kHz) to the cancer cells. Notably, experiments show that the combination of ABT-751 and the alternating electric field produces synergistic results for certain glioblastoma cell lines.

### CROSS REFERENCE TO RELATED APPLICATIONS

This application claims the benefit of U.S. Provisional Application 62/741,791, filed Oct. 5, 2018, which is incorporated herein by reference in its entirety.

### BACKGROUND

Tumor Treating Fields (TTFields) are an effective anti-neoplastic treatment modality delivered via non-invasive application of low intensity, intermediate frequency, alternating electric fields. TTFields exert directional forces on polar microtubules and interfere with the normal assembly of the mitotic spindle. Such interference with microtubule dynamics results in abnormal spindle formation and subsequent mitotic arrest or delay. Cells can die while in mitotic arrest or progress to cell division leading to the formation of either normal or abnormal aneuploid progeny. The formation of tetraploid cells can occur either due to mitotic exit through slippage or can occur during improper cell division. Abnormal daughter cells can die in the subsequent interphase, can undergo a permanent arrest, or can proliferate through additional mitosis where they will be subjected to further TTFields assault. Giladi M. et al. Sci Rep. 2015; 5:18046.

TTFields therapy is delivered using a wearable and portable device (Optune®). The delivery system includes an electric field generator, four adhesive patches (non-invasive, insulated transducer arrays), rechargeable batteries, and a carrying case. The transducer arrays are applied to the skin and are connected to the device and battery. The therapy is designed to be worn for as many hours as possible throughout the day and night.

In the preclinical setting, TTFields can be applied in vitro using, for example, the Inovitro™ TTFields lab bench system. Inovitro™ includes a TTFields generator and base plate containing 8 ceramic dishes per plate. Cells are plated on round cover slips placed inside each dish. TTFields are applied using two perpendicular pairs of transducer arrays insulated by a high dielectric constant ceramic in each dish. The orientation of the TTFields in each dish is switched 90° every 1 second.

### SUMMARY OF THE INVENTION

Aspects described herein provide methods for treating cancer with a combination of TTFields and ABT-751 (i.e., N-[2-(4-hydroxyanilino)pyridin-3-yl]-4-methoxybenzenesulfonamide). Notably, the combination of ABT-751 and TTFields provides a synergistic result for certain types of glioblastoma.

One aspect of the invention is directed to a first method of reducing viability of cancer cells. The first method comprises administering ABT-751 to the cancer cells; and applying an alternating electric field to the cancer cells. The alternating electric field has a frequency between 100 and 400 kHz.

In some instances of the first method, at least a portion of the applying step is performed simultaneously with at least a portion of the administering step.

In some instances of the first method, the ABT-751 comprises a pharmaceutically acceptable carrier.

In some instances of the first method, the applying step has a duration of at least 72 hours.

In some instances of the first method, the applying step has a duration of at least 8 hours.

In some instances of the first method, the applying step comprises applying the alternating electric field for at least three intervals of at least 16 hours each, with a break between each of the at least three intervals during which alternating electric fields are not applied.

In some instances of the first method, the frequency of the alternating electric field is 200 kHz.

In some instances of the first method, the ABT-751 is administered to the cancer cells at a therapeutically effective concentration, and the alternating electric field has a field strength of at least 1 V/cm in at least some of the cancer cells.

In some instances of the first method, the cancer cells comprise glioblastoma cells.

Another aspect of the invention is directed to a second method of reducing viability of cancer cells. The second method comprises administering a microtubule poison to the cancer cells; and applying an alternating electric field to the cancer cells. The alternating electric field has a frequency between 100 and 400 kHz.

In some instances of the second method, at least a portion of the applying step is performed simultaneously with at least a portion of the administering step.

In some instances of the second method, the microtubule poison comprises a pharmaceutically acceptable carrier.

In some instances of the second method, the applying step has a duration of at least 72 hours.

In some instances of the second method, the applying step has a duration of at least 8 hours.

In some instances of the second method, the applying step comprises applying the alternating electric field for at least three intervals of at least 16 hours each, with a break between each of the at least three intervals during which alternating electric fields are not applied.

In some instances of the second method, the frequency of the alternating electric field is 200 kHz.

In some instances of the second method, the microtubule poison is administered to the cancer cells at a therapeutically effective concentration, and the alternating electric field has a field strength of at least 1 V/cm in at least some of the cancer cells.

In some instances of the second method, the cancer cells comprise glioblastoma cells.

### BRIEF DESCRIPTION OF THE DRAWINGS

FIG. 1 shows how the growth of A172 cells is affected by TTFields alone and in combination with ABT-751.

FIG. 2 shows how colony formation of A172 cells is affected by TTFields alone and in combination with ABT-751.

FIG. 3 shows how the growth of U87 cells is affected by TTFields alone and in combination with ABT-751.

FIG. 4 shows how colony formation of U87 cells is affected by TTFields alone and in combination with ABT-751.

FIG. 5 depicts the results of cell cycle analysis by flow cytometry.

FIG. 6 depicts an example system for applying TTFields to a person's brain.

### DESCRIPTION OF THE PREFERRED EMBODIMENTS

The term “treating” refers to ameliorating, inhibiting, reducing growth, inhibiting metastases, and prescribing medication to do the same.

The term “reducing viability of cancer cells” as used herein, refers to reducing the growth, proliferation, or survival of the cancer cells.

The term “therapeutically effective concentration,” as used herein, refers to a concentration sufficient to achieve its intended purpose (e.g., treatment of cancer, treatment of glioblastoma).

Although the treatment of human malignancies has improved dramatically, the treatment options for glioblastoma (GBM) are still limited. A new treatment modality for GBM is tumor treating fields (TTFields), which is an anti-neoplastic treatment modality that creates alternating electric fields that are delivered to the tumor by insulated transducer arrays. Efficacy of TTFields is attributed to mitotic spindle disruption, which eventually leads to improper chromosome segregation and mitotic catastrophe. A phase 3 clinical trial has demonstrated effectiveness of TTFields during maintenance treatment with the DNA alkylating agent temozolomide (TMZ), especially in patients responding well to TMZ. Since the DNA damaging effects of TMZ mainly occur during DNA replication, the inventors believe that this synergistic response could be attributed to effects of TTFields during interphase.

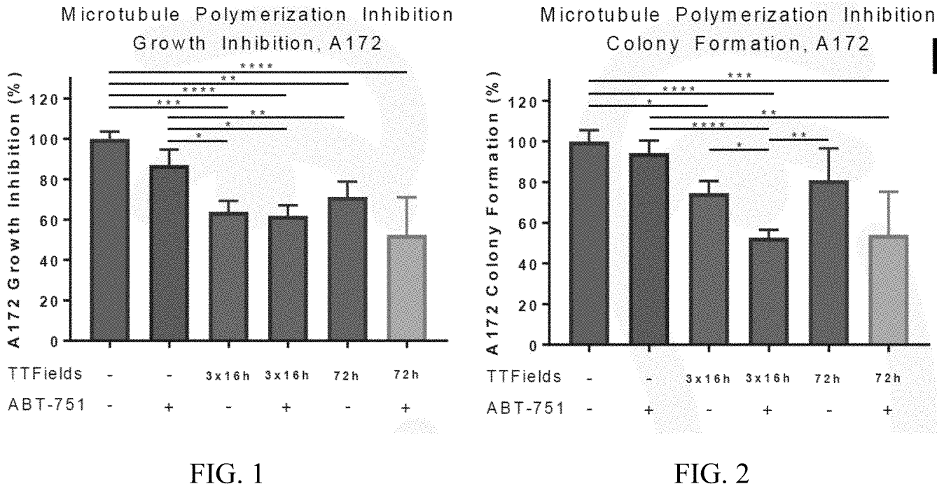

FIG. 1 shows how the growth of A172 cells is affected by TTFields (200 kHz; 3.3 V/cm) alone and in combination with ABT-751 over the course of 72 hours. For the first two bars, both without (−) and with (+) ABT-751, TTFields were not applied. For the next two bars, both without (−) and with (+) ABT-751, TTFields were applied for three 16 hour intervals, and each of those 16 hour intervals was followed by an 8 hour interval during which TTFields were not applied. For the next two bars, both without (−) and with (+) ABT-751, TTFields were applied continuously for 72 hours.

FIG. 2 shows how colony formation of A172 cells is affected by TTFields (200 kHz; 3.3V/cm) alone and in combination with ABT-751 over the course of 72 hours. For the first two bars, both without (−) and with (+) ABT-751, TTFields were not applied. For the next two bars, both without (−) and with (+) ABT-751, TTFields were applied for three 16 hour intervals, and each of those 16 hour intervals was followed by an 8 hour interval during which TTFields were not applied. For the next two bars, both without (−) and with (+) ABT-751, TTFields were applied continuously for 72 hours.

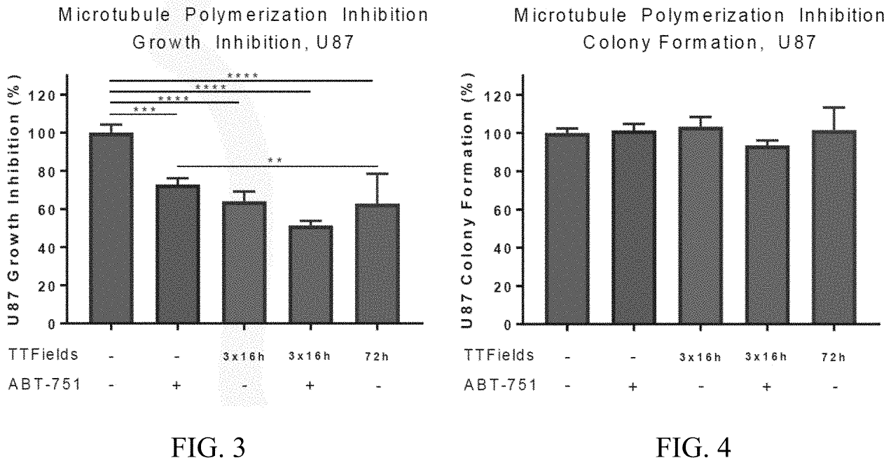

FIG. 3 shows how the growth of U87 cells is affected by TTFields (200 kHz; 3.3 V/cm) alone and in combination with ABT-751 over the course of 72 hours. For the first two bars, both without (−) and with (+) ABT-751, TTFields were not applied. For the next two bars, both without (−) and with (+) ABT-751, TTFields were applied for three 16 hour intervals, and each of those 16 hour intervals was followed by an 8 hour interval during which TTFields were not applied. For the next two bars, both without (−) and with (+) ABT-751, TTFields were applied continuously for 72 hours.

FIG. 4 shows how colony formation of U87 cells is affected by TTFields (200 kHz; 3.3V/cm) alone and in combination with ABT-751 over the course of 72 hours. For the first two bars, both without (−) and with (+) ABT-751, TTFields were not applied. For the next two bars, both without (−) and with (+) ABT-751, TTFields were applied for three 16 hour intervals, and each of those 16 hour intervals was followed by an 8 hour interval during which TTFields were not applied. For the next two bars, both without (−) and with (+) ABT-751, TTFields were applied continuously for 72 hours.

Collectively, FIGS. 1-4 show the following: (a) TTFields inhibit growth of both A172 (FIG. 1) and U87 cells (FIG. 3) and lowers clonogenic survival of A172 cells (FIG. 2); (b) TTFields synergizes with the microtubule poison ABT-751 (1 μM, 8 hours) in A172 (FIGS. 1 and 2), but not in U87 (FIGS. 3 and 4); and (c) short treatment interruptions of eight hours (3×16 hours) do not affect treatment response (FIGS. 1-4). Data are mean±SEM; n≥5; Statistical significance was determined by one-way ANOVA; Tukey-corrected for multiple comparisons; *p<0.05, **p<0.01, ***p<0.001, ****p<0.0001.

Most notably, a synergistic effect between TTFields and ABT-751 was observed for the A172 cells.

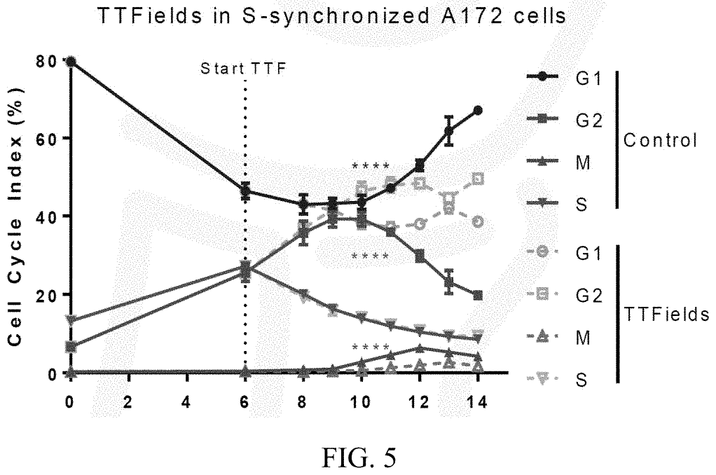

FIG. 5 depicts the results of cell cycle analysis by flow cytometry. These results reveal that TTFields treatment in S-synchronized A172 cells cause a significant accumulation in G2, leading to delayed mitotic entry and, subsequently, lower entry into G1. Data are mean±SEM; n≥4; Statistical significance was determined by testing whether one third-order polynomial fit adequately fits all data sets; Tukey-corrected for multiple comparisons; *p<0.05, **p<0.01, ***p<0.001, ****p<0.0001.

In all the experiments described herein, TTFields (200 kHz; 3.3V/cm) were applied using an Inovitro™ system.

The data described above in connection with FIGS. 1-5 elucidate the effects of TTFields on the cell cycle and design sensitizing strategies that can be implemented using TTFields. The data reveals that (1) TTFields effectively delay growth and decrease clonogenic survival of GBM cells; (2) TTFields synergizes with the microtubule poison ABT-751; and (3) TTFields cause G2 arrest. Without being bound by this theory, this may be through induction of DNA damage during interphase. Notably, equal effectiveness was found in the interrupted treatment schedule (i.e., when TTFields were applied for three 16 hour intervals interrupted by 8 hour breaks) as compared to constant treatment (i.e., when TTFields were applied continuously for 72 hours).

These results establish that the viability of cancer cells can be reduced by administering ABT-751 to the cancer cells and applying an alternating electric field with a frequency between 100 and 400 kHz to the cancer cells.

In the in vitro context, the administering of the ABT-751 to the cancer cells occurs continuously from a first time (t₁) when the ABT-751 is introduced into the container that is holding the cancer cells until such time (t₂) as the ABT-751 is removed or exhausted. As a result, if TTFields are applied to the cancer cells between t₁ and t₂, the applying step will be simultaneous with at least a portion of the administering step.

While the in vitro experiments described above were performed using the frequencies, field intensities, and durations noted above, those parameters may be varied. For example, the frequency could be between 100 and 400 kHz, the electric field intensity could be between 0.5 and 5 V/cm; and the duration could be anything longer than 8 hours. Moreover, while the experiments were performed using a specific microtubule poison (i.e., ABT-751), a different microtubule poison could be substituted for the ABT-751.

In the in vitro experiments using the Inovitro™ system described herein, the direction of the TTFields was switched at one second intervals between two perpendicular directions. But in alternative embodiments, the direction of the TTFields can be switched at a faster rate (e.g., at intervals between 1 and 1000 ms) or at a slower rate (e.g., at intervals between 1 and 100 seconds).

In the in vitro experiments using the Inovitro™ system described herein, the direction of the TTFields was switched between two perpendicular directions by applying an AC voltage to two pairs of electrodes that are disposed 90° apart from each other in 2D space in an alternating sequence. But in alternative embodiments the direction of the TTFields may be switched between two directions that are not perpendicular by repositioning the pairs of electrodes, or between three or more directions (assuming that additional pairs of electrodes are provided). For example, the direction of the TTFields may be switched between three directions, each of which is determined by the placement of its own pair of electrodes. Optionally, these three pairs of electrodes may be positioned so that the resulting fields are disposed 90° apart from each other in 3D space. In other alternative embodiments, the electrodes need not be arranged in pairs. See, for example, the electrode positioning described in U.S. Pat. No. 7,565,205, which is incorporated herein by reference. In other alternative embodiments, the direction of the field remains constant.

In the in vitro experiments using the Inovitro™ system described herein, the electrical field was capacitively coupled into the culture because the Inovitro™ system uses conductive electrodes disposed on the outer surface of the dish sidewalls, and the ceramic material of the sidewalls acts as a dielectric. But in alternative embodiments, the electric field could be applied directly to the cells without capacitive coupling (e.g., by modifying the Inovitro™ system configuration so that the conductive electrodes are disposed on the sidewall's inner surface instead of on the sidewall's outer surface).

The methods described herein can also be applied in the in vivo context by applying the TTFields to a target region of a live subject's body. This may be accomplished, for example, by positioning electrodes on or below the subject's skin so that application of an AC voltage between selected subsets of those electrodes will impose the TTFields in the target region of the subject's body.

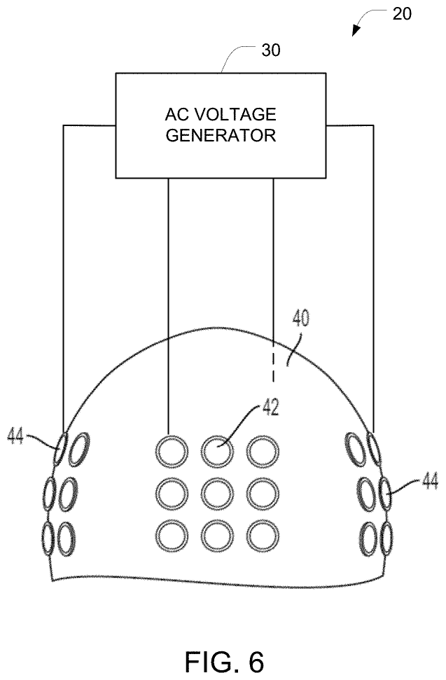

FIG. 6 depicts an example system 20 for applying TTFields to a person's brain (e.g., to treat glioblastoma). The system 20 includes an AC voltage generator 30, a first set of electrodes 44 positioned on the right and left side of the head, and a second set of electrodes 42 positioned on the front and back of the head. (Because FIG. 6 depicts the front view of the scalp 40, the electrodes 42 that are positioned on the back of the head are not visible in this view.) In the illustrated embodiment, each of the electrodes 42, 44 includes nine circular elements that are wired in parallel. But in alternative embodiments, a different number of elements and/or elements with different shapes may be used, depending on the anatomical location where the electrodes will be positioned.

To use this system, the first set of electrodes 44 is applied to the subject's body (i.e., on the right and left sides of the head in the illustrated embodiment). The first set of electrodes 44 is positioned with respect to the target region so that application of an AC voltage between the electrodes 44 will impose TTFields with a first orientation (i.e., right to left in the illustrated embodiment) through the target tissue (i.e., the brain in the illustrated embodiment). The second set of electrodes 42 is also applied to the subject's body (i.e., on the front and back of the head in the illustrated embodiment). The second set of electrodes is positioned with respect to the target region so that application of an AC voltage between the electrodes 42 will impose TTFields with a second orientation through the tissue (i.e., front to back in the illustrated embodiment). The first orientation and the second orientation are different (and are roughly perpendicular in the illustrated embodiment).

After the first and second set of electrodes 42, 44 have been applied to the subject's body, the AC voltage generator 30 repeats the following steps in an alternating sequence: (a) applying a first AC voltage between the electrodes of the first set 44, such that TTFields with the first orientation is imposed through the tissue and (b) applying a second AC voltage between the electrodes of the second set 42, such that TTFields with the second orientation is imposed through the tissue.

In some embodiments, all the electrodes are positioned on the subject's body (as depicted in FIG. 6); in other embodiments, all the electrodes may be implanted in the subject's body (e.g., just beneath the subject's skin, or in the vicinity of the organ being treated); and in other embodiments, some of the electrodes are positioned on the subject's skin and the rest of the electrodes are implanted in the subject's body.

In some embodiments, the electrodes are capacitively coupled to the subject's body (e.g., by using electrodes that include a conductive plate and also have a dielectric layer disposed between the conductive plate and the subject's body). But in alternative embodiments, the dielectric layer may be omitted, in which case the conductive plates would make direct contact with the subject's body.

Optionally, thermal sensors (not shown) may be included at the electrodes, and the AC voltage generator 30 can be configured to decrease the amplitude of the AC voltages that are applied to the electrodes if the sensed temperature at the electrodes gets too high.

Note that while FIG. 6 depicts an embodiment in which the TTFields are applied to the brain, the TTFields may be applied to different portions of a subject's body as described above in alternative embodiments.

In the in vivo context, the administering of the ABT-751 to the subject may be performed using any of a variety of approaches including but not limited to intravenously, orally, subcutaneously, intrathecal, intraventricularly, and intraperitonealy. And the application of the TTFields to the cancer cells may be performed using the Novocure Optune® system or a variant thereof that operates at a different frequency. In the in vivo context, the administering of the ABT-751 to the cancer cells can occur continuously from a first time (t₁) when the ABT-751 is circulating in the patient's body (e.g., after administering it systemically) or introduced into the vicinity of the cancer cells until such time (t₂) as the ABT-751 is eliminated from the patient's body or exhausted. As a result, if TTFields are applied to the cancer cells between t₁ and t₂, the applying step will be simultaneous with at least a portion of the administering step.

Note that any of the parameters for this in vivo embodiment (e.g., frequency, field strength, duration, direction-switching rate, and the placement of the electrodes) may be varied as described above in connection with the in the vitro embodiments.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of reducing viability of cancer cells, the method comprising:
administering ABT-751 to the cancer cells; and
applying an alternating electric field to the cancer cells, the alternating electric field having a frequency between 100 and 400 kHz.

2. The method of claim 1, wherein at least a portion of the applying step is performed simultaneously with at least a portion of the administering step.

3. The method of claim 1, wherein the ABT-751 comprises a pharmaceutically acceptable carrier.

4. The method of claim 1, wherein the applying step has a duration of at least 72 hours.

5. The method of claim 1, wherein the applying step has a duration of at least 8 hours.

6. The method of claim 1, wherein the applying step comprises applying the alternating electric field for at least three intervals of at least 16 hours each, with a break between each of the at least three intervals during which alternating electric fields are not applied.

7. The method of claim 1, wherein the frequency of the alternating electric field is 200 kHz.

8. The method of claim 1, wherein the ABT-751 is administered to the cancer cells at a therapeutically effective concentration, and wherein the alternating electric field has a field strength of at least 1 V/cm in at least some of the cancer cells.

9. The method of claim 1, wherein the cancer cells comprise glioblastoma cells.
